(19) United States
(12) Patent Application Publication
(10) Pub. No.: US 2012/0027876 A1
(43) Pub. Date: Feb. 2, 2012

(54) Composition and Method for the Topical Treatment of Dermatitis

(76) Inventor: Sara Beth Ford, Hamilton, MS (US)

(21) Appl. No.: 12/844,770

(22) Filed: Jul. 27, 2010

(51) Int. Cl. A61K 33/08; A61P 17/08; A61P 17/06

(52) U.S. Cl. 424/690

(57) ABSTRACT

A composition for topical treatment of dermatitis is provided. The composition includes one or more anti-histamines or a pharmaceutically acceptable salt thereof; one or more polysaccharides; and one or more Group 1, 2, or 13 metal hydroxides. The dermatitis may be a poison ivy, poison oak, poison sumac, diaper rash, eczema, lichen simplex chronicus, rashes, dermatoses, seborrheic dermatitis, psoriasis, atopic dermatitis, or the combination thereof. A method of treatment of dermatitis is also provided.

### BACKGROUND OF THE INVENTION

Contact dermatitis is an inflammation of the skin and is an acute or chronic condition resulting from irritation by or sensitization to, some substance in the environment. For example, contact with poison ivy, poison oak, and poison sumac, may cause the formation of a rash. A variety of methods exist for treating contact dermatitis, including, for example, topical corticosteroids, aluminum acetate, calamine lotion, oral anti-histamines, and systemic corticosteroids. None of these therapies provide complete relief so a combination of therapies is for an existing condition.

Another type of common contact dermatitis is diaper rash. Diaper rash is the most common form of contact dermatitis in childhood. Occlusion of the groin with diapers allows increased concentration of moisture in the area eventually leading to the breakdown of the underlying skin. Although diaper rash is usually mild and transient, bacteria or fungi sometimes invade the damaged skin, causing a severe diaper rash requiring medical evaluation and treatment. Many ointments and powders exist on the market for treating and preventing diaper dermatitis, but most function by merely forming a barrier between the skin and expressed feces.

There are several other skin diseases that act in a similar fashion to contact dermatitis. These include eczema, lichen simplex chronicus, rashes, dermatoses, seborrheic dermatitis, psoriasis, atopic dermatitis, and others. All of these conditions can present with a rash that can become moist, weeping, and quite irritated. The rashes can also become secondarily infected.

What is needed is a topically applied treatment that provides quick relief to a wide variety of dermatitis including poison ivy, poison oak, poison sumac, diaper rash, eczema, lichen simplex chronicus, rashes, dermatoses, seborrheic dermatitis, psoriasis, atopic dermatitis, and the like.

### SUMMARY OF THE INVENTION

The present invention provides a composition that provides quick and exceptional treatment for a wide variety of dermatitis. The composition, when applied to affected skin, leaves a light film that provides protection from the external environment. The light film does not smother the affected skin like the currently available zinc-oxide products do. Further, the skin is allowed to breathe to promote the healing process. The composition dries out the irritants to shorten the duration of the rash. The composition remains on the skin until it is removed by gentle washing with warm water. Typically, the composition significantly improves red irritated skin within 24 hours.

The composition is of great value to mothers of infants with diaper rash, to nursing home residents with skin infections from diarrhea and urine rashes, to patients suffering from poison ivy and poison oak by sealing the oils and relieving the itch, and any irritation resulting in red inflamed skin.

The present invention provides a composition for topical treatment of dermatitis. The composition includes: one or more anti-histamines or a pharmaceutically acceptable salt thereof; one or more polysaccharides; and one or more Group 1, 2, or 13 metal hydroxides, wherein the dermatitis is poison ivy, poison oak, poison sumac, diaper rash, eczema, lichen simplex chronicus, rashes, dermatoses, seborrheic dermatitis, psoriasis, atopic dermatitis, or the combination thereof.

In one embodiment, the composition includes a solution, spray, lotion, gel, cream, or ointment. In another embodiment, the composition includes a gel.

In one embodiment, the one or more anti-histamines or a pharmaceutically acceptable salt thereof each comprise brompheniramine, chlorpheniramine debrompheniramine, dexchlorpheniramine, carbinoxamine, clemastine, diphenhydramine, pyrilamine, tripelennamine, tripolidine, methdilazine, bromodiphenhydramine, promethazine, azatadine, cyproheptadine, diphenylpyraline, doxylamine, trimeprazine, phenindamine, ketotifen, hydroxyzine, tazifylline, temelastine, meclizine, acrivastine, setastine, oxatomide, mequitazine, levocabastine, lodoxamide, rocastine, phenindamine, azelastine, and ebastine, fexofenadine, loratadine, descarboethoxy loratadine, astemizole, norastemizole, desmethylastemizole, cetirizine, acrivastine, and temelastine, or a combination thereof.

In another embodiment, the one or more anti-histamines or a pharmaceutically acceptable salt thereof each comprise diphenhydramine hydrochloride.

In yet another embodiment, the one or more polysaccharides each comprise starch, a gum, or a combination thereof. In one embodiment, the starch includes corn starch, potato starch, wheat starch, tapioca starch, cassava starch, arrowroot starch, arracacha starch, buckwheat starch, barley starch, oat starch, millet starch, rye starch, banana starch, breadfruit starch, canna starch, colacassia starch, katakuri starch, kudzu starch, malanga starch, oca starch sago starch, sorghum starch, sweet potato starch, taro starch, water chestnut starch, yam starch, fava starch, lentils starch, mung bean starch, pea starch, or a combination thereof. In another embodiment, the starch is corn starch.

In yet another embodiment, the gum includes xanthan gum, gum Arabic, guar gum, red gum, gum acacia, sweet gum, black gum, kauri gum, or a combination thereof. In one embodiment, the gum is xanthan gum.

In another embodiment, the one or more Group 1, 2, or 13 metal hydroxides each comprise lithium hydroxide, sodium hydroxide, potassium hydroxide, magnesium hydroxide, calcium hydroxide, boron hydroxide, aluminum hydroxide, or a combination thereof. In yet another embodiment, the one or more Group 1, 2, or 13 metal hydroxides comprise the combination of magnesium hydroxide and aluminum hydroxide.

In one embodiment, the composition further includes simethicone. In another embodiment, the composition includes from about 0.1 to about 10.0 percent by weight of one or more anti-histamines or a pharmaceutically acceptable salt thereof; from about 0.1 to about 70.0 percent by weight of one or more polysaccharides; and from about 0.1 to about 20.0 percent by weight one or more Group 1, 2, or 13 metal hydroxides.

The present invention provides a composition for topical treatment of dermatitis. The composition includes: diphenhydramine hydrochloride; corn starch; xanthan gum powder; aluminum hydroxide; and magnesium hydroxide; wherein the dermatitis is poison ivy, poison oak, poison sumac, diaper rash, eczema, lichen simplex chronicus, rashes, dermatoses, seborrheic dermatitis, psoriasis, atopic dermatitis, or the combination thereof.

In one embodiment, the composition includes: from about 0.1 to about 10.0 percent by weight of diphenhydramine hydrochloride; from about 5.0 to about 70.0 percent by weight of corn starch; from about 0.1 to about 10.0 percent by weight of xanthan gum powder; from about 0.1 to about 10.0 percent by weight of aluminum hydroxide; and from about 0.1 to about 10.0 percent by weight of magnesium hydroxide.

In another embodiment, the composition includes: from about 0.1 to about 5.0 percent by weight of diphenhydramine hydrochloride; from about 20.0 to about 50.0 percent by weight of corn starch; from about 0.1 to about 5.0 percent by weight of xanthan gum powder; from about 1.0 to about 5.0 percent by weight of aluminum hydroxide; and from about 1.0 to about 5.0 percent by weight of magnesium hydroxide.

In yet another embodiment, the composition includes: from about 0.5 to about 1.0 percent by weight of diphenhydramine hydrochloride; from about 35.0 to about 40.0 percent by weight of corn starch; from about 0.5 to about 1.5 percent by weight of xanthan gum powder; from about 2.0 to about 3.0 percent by weight of aluminum hydroxide; and from about 2.0 to about 3.0 percent by weight of magnesium hydroxide.

In one embodiment, the composition includes: about 0.8 percent by weight of diphenhydramine hydrochloride; about 37.0 percent by weight of corn starch; about 1.0 percent by weight of xanthan gum powder; about 2.5 percent by weight of aluminum hydroxide; and about 2.5 percent by weight of magnesium hydroxide.

The present invention provides a composition for topical treatment of dermatitis. The composition consisting essentially of: diphenhydramine hydrochloride; corn starch; xanthan gum powder; aluminum hydroxide; and magnesium hydroxide; wherein the dermatitis is poison ivy, poison oak, poison sumac, diaper rash, eczema, lichen simplex chronicus, rashes, dermatoses, seborrheic dermatitis, psoriasis, atopic dermatitis, or the combination thereof.

The present invention provides a composition for topical treatment of dermatitis. The composition consisting essentially of about 0.8 percent by weight of diphenhydramine hydrochloride; about 37.0 percent by weight of corn starch; about 1.0 percent by weight of xanthan gum powder; about 2.5 percent by weight of aluminum hydroxide; and about 2.5 percent by weight of magnesium hydroxide, wherein the dermatitis is poison ivy, poison oak, poison sumac, diaper rash, eczema, lichen simplex chronicus, rashes, dermatoses, seborrheic dermatitis, psoriasis, atopic dermatitis, or the combination thereof.

The present invention provides a method of treating dermatitis. The method includes: applying to a skin suffering from dermatitis a composition including one or more anti-histamines or a pharmaceutically acceptable salt thereof; one or more polysaccharides; and one or more Group 1, 2, or 13 metal hydroxides, wherein the dermatitis is poison ivy, poison oak, poison sumac, diaper rash, eczema, lichen simplex chronicus, rashes, dermatoses, seborrheic dermatitis, psoriasis, atopic dermatitis, or the combination thereof.

The present invention provides a method of treating dermatitis. The method includes: applying to a skin suffering from dermatitis a composition for topical treatment of dermatitis including: diphenhydramine hydrochloride; corn starch; xanthan gum powder; aluminum hydroxide; and magnesium hydroxide; wherein the dermatitis is poison ivy, poison oak, poison sumac, diaper rash, eczema, lichen simplex chronicus, rashes, dermatoses, seborrheic dermatitis, psoriasis, atopic dermatitis, or the combination thereof.

### DETAILED DESCRIPTION OF THE INVENTION

The present invention provides a composition that provides quick and exceptional treatment for a wide variety of dermatitis. The composition, when applied to affected skin, leaves a light film that provides protection from the external environment. The light film does not smother the affected skin like the currently available zinc-oxide products do. Further, the skin is allowed to breathe to promote the healing process. The composition dries out the irritants to shorten the duration of the rash. The composition remains on the skin until it is removed by gentle washing with warm water. Typically, the composition significantly improves red irritated skin within 24 hours.

The composition is of great value to mothers of infants with diaper rash, to nursing home residents with skin infections from diarrhea and urine rashes, to patients suffering from poison ivy and poison oak by sealing the oils and relieving the itch, and any irritation resulting in red inflamed skin.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Unless otherwise indicated, the words and phrases presented in this document have their ordinary meanings to one of skill in the art. Such ordinary meanings can be obtained by reference to their use in the art and by reference to general and scientific dictionaries, for example, Webster's Third New International Dictionary, Merriam-Webster Inc., Springfield, Mass., 1993 and The American Heritage Dictionary of the English Language, Houghton Mifflin, Boston Mass., 1981.

The following explanations of certain terms are meant to be illustrative rather than exhaustive. These terms have their ordinary meanings given by usage in the art and in addition include the following explanations.

As used herein, the term “about” refers to a variation of 10 percent of the value specified; for example about 50 percent carries a variation from 45 to 55 percent.

As used herein, the term “and/or” refers to any one of the items, any combination of the items, or all of the items with which this term is associated.

As used herein, the singular forms “a,” “an,” and “the” include plural reference unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

As used herein, the term “administration” refers to a method of placing a device to a desired site. The placing of a device can be by any pharmaceutically accepted means such as by swallowing, retaining it within the mouth until the drug has been dispensed, placing it within the buccal cavity, inserting, implanting, attaching, etc. These and other methods of administration are known in the art.

As used herein, the term “anti-histamine” refers to Histamine H₁ receptor antagonists.

As used herein, the term “aqueous medium” refers to a liquid medium composed largely, but not necessarily exclusively, of water. Other components may also be present, such as salts, co-solvents, buffers, stabilizers, dispersants, colorants and the like.

As used herein, the term “atopic dermatitis” refers to a disorder that involves itching eczema as a principal lesion which undergoes repeated exacerbation and remission; this is highly likely to develop in individuals predisposed to atopy.

As used herein, the term “derivative” of a compound refers to a chemically modified compound wherein the chemical modification takes place at one or more functional groups of the compound and /or on an aromatic, alicyclic, or heterocyclic structures, when present. The derivative however is expected to retain the pharmacological activity of the compound from which it is derived.

As used herein, the term “diaper rash” refers to a response indicated by the occurrence on diapered epidermis of what is clinically defined as either erythema or dermatitis, or both, where persistent redness, dryness, pustules and/or other symptoms associated with such conditions may arise.

As used herein, the term “an effective amount” refers to an amount sufficient to effect beneficial or desired results. An effective amount can be administered in one or more administrations, applications, or dosages. Determination of an effective amount for a given administration is well within the ordinary skill in the pharmaceutical arts.

As used herein, the term “eczema” refers to the swelling of the outer skin.

As used herein, the term “Group 1, 2, or 13 metal hydroxides” refer to a compound of the formula Me(OH), Me(OH)₂, or Me(OH)₄, wherein Me is a metal selected from the Group 1, 2, or 13 listed in the International Union of Pure and Applied Chemistry (IUPAC) Periodic Table, June 2007 Edition.

As used herein, the term “infection” refers to the invasion of the host by germs that reproduce and multiply, causing disease by local cell injury, release of poisons, or germ-antibody reaction in the cells. The infection can be in a mammal (e.g., human).

As used herein, the terms “include,” “for example,” “such as,” and the like are used illustratively and are not intended to limit the present invention.

As used herein, the terms “optional” or “optionally” mean that the subsequently described event or condition may but need not occur, and that the description includes instances where the event or condition occurs and instances in which it does not.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio. Several pharmaceutically acceptable ingredients are known in the art and official publications such as The United States Pharmacopeia describe the analytical criteria to assess the pharmaceutical acceptability of numerous ingredients of interest.

As used herein, the term “pharmaceutically acceptable salts” refers to ionic compounds, wherein a parent non-ionic compound is modified by making acid or base salts thereof. Examples of pharmaceutically acceptable salts include mineral or organic acid salts of basic residues such as amines; alkali or organic salts of acidic residues such as carboxylic acids; and the like. The pharmaceutically acceptable salts include conventional non-toxic salts and quaternary ammonium salts of the parent compound formed, for example, from non-toxic inorganic or organic acids. Non-toxic salts can include those derived from inorganic acids such as hydrochloric, hydrobromic, hydroiodic, sulfuric, sulfamic, phosphoric, nitric and the like. Salts prepared from organic acids can include those such as acetic, 2-acetoxybenzoic, ascorbic, benzenesulfonic, benzoic, citric, ethanesulfonic, ethane disulfonic, formic, fumaric, gentisinic, glucaronic, gluconic, glutamic, glycolic, hydroxymaleic, isethionic, isonicotinic, lactic, maleic, malic, mesylate or methanesulfonic, oxalic, pamoic (1,1′-methylene-bis-(2-hydroxy-3-naphthoate)), pantothenic, phenylacetic, propionic, salicylic, sulfanilic, toluenesulfonic, stearic, succinic, tartaric, bitartaric, and the like. Certain compounds can form pharmaceutically acceptable salts with various amino acids. For a review on pharmaceutically acceptable salts, see, e.g., Berge et al., J. Pharm. Sci. 1977, 66(1), 1-19, which is incorporated herein by reference.

The pharmaceutically acceptable salts of the compounds described herein can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of many suitable salts are found in Remington: The Science and Practice of Pharmacy, 21^{st} edition, Lippincott, Williams & Wilkins, (2005).

As used herein, the term “polysaccharide” refers to polymers of any length and dimensions comprising monosaccharide residues linked glycosidically in branched or unbranched chains.

As used herein, the term “psoriasis” refers to an inborn skin disorder typically accompanied by red patches with thick, dry, silvery scales and sometimes swelling of small joints.

As used herein, the terms “prevent,” “preventative,” “prevention,” “protect,” and “protection” refer to medical procedures that keep the malcondition from occurring in the first place. The terms mean that there is no or a lessened development of disease or disorder where none had previously occurred, or no further disorder or disease development if there had already been development of the disorder or disease.

As used herein, the term “pruritic symptoms” refers to those symptoms which involve circumscribed or generalized itching and associated inflammations on the skin and mucous membranes. Examples include scabies, urticaria, eczema, xerosis (senile xeroderma and asteatotic eczema), psoriasis, dermal pruritus, and prurigo.

As used herein, the term “rash” refers to a response indicated by the occurrence on epidermis of what is clinically defined as erythema, dermatitis, psoriasis, or any of several other skin conditions where persistent redness, dryness, papules, pustules and/or other symptoms associated with such conditions may arise.

As used herein, the term “seborrheic dermatitis” refers to a common, long-term, inflammatory skin disease marked by dry or moist greasy scales and yellowish crusts.

As used herein, the term “simethicone” refers to the United States Pharmacopoeia (USP XXII) definition, which is a mixture of fully methylated linear siloxane polymers containing repeating units of polydimethylsiloxane stabilized with trimethylsiloxy end-blocking units, and silicon dioxide.

As used herein, the terms “treating” or “treat” or “treatment” refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. As used herein, the term “treatment,” covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used herein, “μg” denotes microgram, “mg” denotes milligram, “g” denotes gram, “μL” denotes microliter, “mL” denotes milliliter, “L” denotes liter, “nM” denotes nanomolar, “μM” denotes micromolar, “mM” denotes millimolar, “M” denotes molar, and “nm” denotes nanometer.

The present invention provides a composition for topical treatment of dermatitis. The composition includes: one or more anti-histamines or a pharmaceutically acceptable salt thereof; one or more polysaccharides; and one or more Group 1, 2, or 13 metal hydroxides. The dermatitis may include, for example, poison ivy, poison oak, poison sumac, diaper rash, eczema, lichen simplex chronicus, rashes, dermatoses, seborrheic dermatitis, psoriasis, atopic dermatitis, or the combination thereof.

The typical composition includes from about 0.1 to about 10.0 percent by weight of one or more anti-histamines or a pharmaceutically acceptable salt thereof; from about 0.1 to about 70.0 percent by weight of one or more polysaccharides; and from about 0.1 to about 20.0 percent by weight of one or more Group 1, 2, or 13 metal hydroxides.

The composition can be in the form of a solution, spray, lotion, gel, cream, or ointment, depending upon the application. In one embodiment, the composition is in the form of a gel.

### Anti-Histamines

A wide variety of anti-histamines or a pharmaceutically acceptable salt thereof can be used in the compositions. Exemplary anti-histamines or a pharmaceutically acceptable salt thereof (and concentrations expressed as a weight percentage of the) include:

Brompheniramine (from about 0.1 to about 10.0 percent by weight, or from about 0.1 to about 5.0 percent by weight, or from about 0.5 to about 1.0 percent by weight):

Chlorpheniramine (from about 0.1 to about 10.0 percent by weight, or from about 0.1 to about 5.0 percent by weight, or from about 0.5 to about 1.0 percent by weight):

Debrompheniramine (from about 0.1 to about 10.0 percent by weight, or from about 0.1 to about 5.0 percent by weight, or from about 0.5 to about 1.0 percent by weight):

Dexchlorpheniramine (from about 0.1 to about 10.0 percent by weight, or from about 0.1 to about 5.0 percent by weight, or from about 0.5 to about 1.0 percent by weight):

Carbinoxamine (from about 0.1 to about 10.0 percent by weight, or from about 0.1 to about 5.0 percent by weight, or from about 0.5 to about 1.0 percent by weight):

Clemastine (from about 0.1 to about 10.0 percent by weight, or from about 0.1 to about 5.0 percent by weight, or from about 0.5 to about 1.0 percent by weight):

Diphenhydramine (from about 0.1 to about 10.0 percent by weight, or from about 0.1 to about 5.0 percent by weight, or from about 0.5 to about 1.0 percent by weight):

Pyrilamine (from about 0.1 to about 10.0 percent by weight, or from about 0.1 to about 5.0 percent by weight, or from about 0.5 to about 1.0 percent by weight):

Tripelennamine (from about 0.1 to about 10.0 percent by weight, or from about 0.1 to about 5.0 percent by weight, or from about 0.5 to about 1.0 percent by weight):

Tripolidine (from about 0.1 to about 10.0 percent by weight, or from about 0.1 to about 5.0 percent by weight, or from about 0.5 to about 1.0 percent by weight):

Methdilazine (from about 0.1 to about 10.0 percent by weight, or from about 0.1 to about 5.0 percent by weight, or from about 0.5 to about 1.0 percent by weight):

Bromodiphenhydramine (from about 0.1 to about 10.0 percent by weight, or from about 0.1 to about 5.0 percent by weight, or from about 0.5 to about 1.0 percent by weight):

Promethazine (from about 0.1 to about 10.0 percent by weight, or from about 0.1 to about 5.0 percent by weight, or from about 0.5 to about 1.0 percent by weight):

Azatadine (from about 0.1 to about 10.0 percent by weight, or from about 0.1 to about 5.0 percent by weight, or from about 0.5 to about 1.0 percent by weight):

Cyproheptadine (from about 0.1 to about 10.0 percent by weight, or from about 0.1 to about 5.0 percent by weight, or from about 0.5 to about 1.0 percent by weight):

Diphenylpyraline (from about 0.1 to about 10.0 percent by weight, or from about 0.1 to about 5.0 percent by weight, or from about 0.5 to about 1.0 percent by weight):

Doxylamine (from about 0.1 to about 10.0 percent by weight, or from about 0.1 to about 5.0 percent by weight, or from about 0.5 to about 1.0 percent by weight):

Trimeprazine (from about 0.1 to about 10.0 percent by weight, or from about 0.1 to about 5.0 percent by weight, or from about 0.5 to about 1.0 percent by weight):

Phenindamine (from about 0.1 to about 10.0 percent by weight, or from about 0.1 to about 5.0 percent by weight, or from about 0.5 to about 1.0 percent by weight):

Ketotifen (from about 0.1 to about 10.0 percent by weight, or from about 0.1 to about 5.0 percent by weight, or from about 0.5 to about 1.0 percent by weight):

Hydroxyzine (from about 0.1 to about 10.0 percent by weight, or from about 0.1 to about 5.0 percent by weight, or from about 0.5 to about 1.0 percent by weight):

Tazifylline (from about 0.1 to about 10.0 percent by weight, or from about 0.1 to about 5.0 percent by weight, or from about 0.5 to about 1.0 percent by weight):

Temelastine (from about 0.1 to about 10.0 percent by weight, or from about 0.1 to about 5.0 percent by weight, or from about 0.5 to about 1.0 percent by weight):

Meclizine (from about 0.1 to about 10.0 percent by weight, or from about 0.1 to about 5.0 percent by weight, or from about 0.5 to about 1.0 percent by weight):

Acrivastine (from about 0.1 to about 10.0 percent by weight, or from about 0.1 to about 5.0 percent by weight, or from about 0.5 to about 1.0 percent by weight):

Setastine (from about 0.1 to about 10.0 percent by weight, or from about 0.1 to about 5.0 percent by weight, or from about 0.5 to about 1.0 percent by weight):

Oxatomide (from about 0.1 to about 10.0 percent by weight, or from about 0.1 to about 5.0 percent by weight, or from about 0.5 to about 1.0 percent by weight):

Mequitazine (from about 0.1 to about 10.0 percent by weight, or from about 0.1 to about 5.0 percent by weight, or from about 0.5 to about 1.0 percent by weight):

Levocabastine (from about 0.1 to about 10.0 percent by weight, or from about 0.1 to about 5.0 percent by weight, or from about 0.5 to about 1.0 percent by weight):

Lodoxamide (from about 0.1 to about 10.0 percent by weight, or from about 0.1 to about 5.0 percent by weight, or from about 0.5 to about 1.0 percent by weight):

Rocastine (from about 0.1 to about 10.0 percent by weight, or from about 0.1 to about 5.0 percent by weight, or from about 0.5 to about 1.0 percent by weight):

Phenindamine (from about 0.1 to about 10.0 percent by weight, or from about 0.1 to about 5.0 percent by weight, or from about 0.5 to about 1.0 percent by weight):

Azelastine (from about 0.1 to about 10.0 percent by weight, or from about 0.1 to about 5.0 percent by weight, or from about 0.5 to about 1.0 percent by weight):

Ebastine (from about 0.1 to about 10.0 percent by weight, or from about 0.1 to about 5.0 percent by weight, or from about 0.5 to about 1.0 percent by weight):

Fexofenadine (from about 0.1 to about 10.0 percent by weight, or from about 0.1 to about 5.0 percent by weight, or from about 0.5 to about 1.0 percent by weight):

Loratadine (from about 0.1 to about 10.0 percent by weight, or from about 0.1 to about 5.0 percent by weight, or from about 0.5 to about 1.0 percent by weight):

Descarboethoxy loratadine (from about 0.1 to about 10.0 percent by weight, or from about 0.1 to about 5.0 percent by weight, or from about 0.5 to about 1.0 percent by weight):

Astemizole (from about 0.1 to about 10.0 percent by weight, or from about 0.1 to about 5.0 percent by weight, or from about 0.5 to about 1.0 percent by weight):

Norastemizole (from about 0.1 to about 10.0 percent by weight, or from about 0.1 to about 5.0 percent by weight, or from about 0.5 to about 1.0 percent by weight):

Desmethylastemizole (from about 0.1 to about 10.0 percent by weight, or from about 0.1 to about 5.0 percent by weight, or from about 0.5 to about 1.0 percent by weight):

Cetirizine (from about 0.1 to about 10.0 percent by weight, or from about 0.1 to about 5.0 percent by weight, or from about 0.5 to about 1.0 percent by weight):

Acrivastine (from about 0.1 to about 10.0 percent by weight, or from about 0.1 to about 5.0 percent by weight, or from about 0.5 to about 1.0 percent by weight): and

Temelastine (from about 0.1 to about 10.0 percent by weight, or from about 0.1 to about 5.0 percent by weight, or from about 0.5 to about 1.0 percent by weight), or a combination thereof.

In one embodiment, the anti-histamine is diphenhydramine hydrochloride.

The compositions may contain each of the above-identified anti-histamines in solution, spray, lotion, cream, gel or ointment form with the anti-histamine selected and the concentration thereof in the composition, depending at least in part, upon the therapeutic application and the vehicle selected.

### Polysaccharides

A wide variety of one or more polysaccharides; can be used in the compositions. Exemplary polysaccharides include, for example, one or more starches, one or more gums, or a combination thereof.

Suitable starches include, for example, starch, potato starch, wheat starch, tapioca starch, cassava starch, arrowroot starch, arracacha starch, buckwheat starch, barley starch, oat starch, millet starch, rye starch, banana starch, breadfruit starch, canna starch, colacassia starch, katakuri starch, kudzu starch, malanga starch, oca starach, sago starch, sorghum starch, sweet potato starch, taro starch, water chestnut starch, yam starch, fava starch, lentils starch, mung bean starch, pea starch, or a combination thereof. In one embodiment, the starch is corn starch.

Suitable gums, include, for example, xanthan gum, gum Arabic, guar gum, red gum, gum acacia, sweet gum, black gum, kauri gum, or a combination thereof. In one embodiment, the gum is xanthan gum.

In one embodiment, the one or more polysaccharides are present in the composition from about 0.1 to about 70.0 percent by weight, or from about 20.0 to about 50.0 percent by weight, or from about 35.0 to about 40.0 percent by weight.

In another embodiment, a combination of a starches and gums are used. The combination of starches and gums are present in the composition from about 0.1 to about 80.0 percent by weight, or from about 20.0 to about 50.0 percent by weight, or from about 35.0 to about 40.0 percent by weight.

In yet another embodiment, the combination of corn starch and xanthan gum is used. The corn starch is present in the composition from about 5.0 to about 70.0 percent by weight of corn starch, or from about 20.0 to about 50.0 percent by weight of corn starch, or from about 35.0 to about 40.0 percent by weight of corn starch, or at about 37.0 percent by weight of corn starch. The xanthan gum is present in the composition from about 0.1 to about 10.0 percent by weight of xanthan gum powder, or from about 20.0 to about 50.0 percent by weight of corn starch, or from about 35.0 to about 40.0 percent by weight of corn starch, or about 37.0 percent by weight of corn starch.

### Metal Hydroxides

A wide variety of one or more Group 1, 2, or 13 metal hydroxides can be used in the compositions. Group 1, 2, or 13 metal hydroxides have the formula Me(OH), Me(OH)₂, or Me(OH)₄, wherein Me is a metal selected from the Group 1, 2, or 13 listed in the International Union of Pure and Applied Chemistry (IUPAC) Periodic Table, June 2007 Edition. Exemplary Group 1, 2, or 13 metal hydroxides include, for example, lithium hydroxide, sodium hydroxide, potassium hydroxide, rubidium hydroxide, caesium hydroxide, magnesium hydroxide, calcium hydroxide, strontium hydroxide, radium hydroxide, boron hydroxide, aluminum hydroxide, gallium hydroxide, indium hydroxide, thallium hydroxide, or combinations thereof.

In one embodiment, the one or more Group 1, 2, or 13 metal hydroxides are present in the composition from about 0.1 to about 20.0 percent by weight.

In one embodiment, a combination of aluminum hydroxide and magnesium hydroxide is present in the composition. In one embodiment, from about 0.1 to about 10.0 percent by weight of aluminum hydroxide and from about 0.1 to about 10.0 percent by weight of magnesium hydroxide is used. In another embodiment, from about 1.0 to about 5.0 percent by weight of aluminum hydroxide and from about 1.0 to about 5.0 percent by weight of magnesium hydroxide is used. In yet another embodiment, from about 2.0 to about 3.0 percent by weight of aluminum hydroxide and from about 2.0 to about 3.0 percent by weight of magnesium hydroxide is used. In still yet another embodiment, from about 2.5 percent by weight of aluminum hydroxide and about 2.5 percent by weight of magnesium hydroxide is used.

### Additional Agents

The composition may also include one or more optional ingredients, for example, anti-acne agents, anti-itch agents, anti-oxidants, anti-microbial agents, anti-fungal agents, non-steroid cosmetic soothing agents, skin conditioning agents, anti-foaming agents, buffers, neutralizing agents, pH adjusting agents, coloring agents, decoloring agents, emollients, emulsifying agents, emulsion stabilizers, viscosity builders, humectants, odorants, preservatives, antioxidants, chemical stabilizers, thickening agents, steroids, organic solvents, water, and combinations thereof.

Suitable anti-acne agents include, for example, keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, including N-acetyl-L-cysteine; lipoic acid; antibiotics and anti-microbials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids and bioflavonoids; bile salts such as scymnol sulfate and its derivatives, deoxycholate, and cholate; abietic acid; adapalene; allantoin; aloe extracts; arbietic acid and its salts; aryl-2,4 dioxo oxazolidine derivatives; Aseboil (Laboratories Serobiologiques, Somerville, N.J.); azaleic acid; barberry extracts; bearberry extracts; belamcanda chinensis; benzoquinolinones; benzoyl peroxide; berberine; Biodermine (Sederma, Brooklyn, N.Y.); bioflavinoids; bisabolol; S-carboxymethyl cysteine; carrot extracts; cassin oil; clove extracts; citral; citronellal; climazole; Completech MBAC-OS (Lipo); Cremogen M82 (Dragoco, Totowa, N.J.); cucumber extracts; dehydroacetic acid and its salts; dehydroeplandersterone salicylate; dichlorophenyl imidazoldioxolan which is commercially available as Completech MBAC-OS (from Lipo, Paterson, N.J.); DL valine and its esters; DMDM hydantoin; Epicutin TT (CLR); erythromycin; escinol; ethyl hexyl monoglyceryl ether; ethyl 2-hydroxy undecanoate; farnesol; farnesol acetate; Geraniol; glabridin; gluconic acid; gluconolactone; glyceryl monocaprate; glycolic acid; grapefruit seed extract; gugu lipid; Hederagenin (Maruzen); hesperitin; hinokitol; hops extract; hydrogenated rosin; 10-hydroxy decanoic acid; ichthyol; interleukin 1 alpha antagonists; iodo-2-propynyl butyl carbamate; Kapilarine (Greentech); ketoconazole; lactic acid; lemon grass oil; Lichochalcone LR15 (Maruzen); linoleic acid; LipacideC8CO (Seppic, Paris, France); lovastatin; 4 methoxysalicylic acid; metronidazole; minocycline; mukurossi; neem seed oil; vitamin B3 compounds (such as niacinamide and nicotinic acid); nisin; 5-octanoyl salicylic acid; octopirox; panthenol; 1-pentadecanol; peonia extract; peppermint extract; phelladendron extract; 2-phenyl-benzothiophene derivatives; phloretin; Phlorogine (Secma); phosphatidyl choline; proteolytic enzymes; quercetin; red sandalwood extract; resorcinol; rosemary extract; rutin; sage extract; salicin; salicylic acid; skull cap extract; siber hegner extract; siberian saxifrage extract; silicol; sodium lauryl sulfate; sodium sulfoacetamide; Sophora Extract (Maruzen); sorbic acid; sulfur; sunder vati extract; tea tree oil; tetracycline; tetra hydroabietic acid; thyme extract; tioxolone; tocopherol; trehalose 6-undecylenoate; 3 tridecene-2-ol; triclosan; tropolone; Unitrienol T27 (Unichem, Gouda, Netherlands); vitamin D3 and its analogs; white thyme oil; willow bark extract; wogonin; Ylang Ylang; zinc glycerolate; zinc linoleate; zinc oxide; zinc pyrithione; zinc sulfate, and combinations thereof.

Suitable antifoaming agents include, for example, cyclomethicone, dimethicone (e.g., dimethicone 350), Simethicone, and combinations thereof.

Suitable anti-itch agents include, for example, Stimutex (Pentapharm), Takanal (Ikeda), Ichthyol (International Sourcing), Oxygenated Glyceryl Triesters (Seporgia), and combinations thereof.

Suitable antioxidants include, for example, tocopheryl acetate, betaglucan, butylated hydroxy toluene (BHT), and combinations thereof.

Suitable anti-microbial and anti-fungal agents may include, for example, beta-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione, clotrimazole, alantolactone, isoalantolactone, alkanet extract (alanine), anise, arnica extract (helenalin acetate and 11,13 dihydrohelenalin), Aspidium extract (phloro, lucinol containing extract), barberry extract (berberine chloride), bay sweet extract, bayberry bark extract (myricitrin), benzalkonium chloride, benzethonium chloride, benzoic acid and its salts, benzoin, benzyl alcohol, blessed thistle, bletilla tuber, bloodroot, bois de rose oil, burdock, butyl paraben, cade oil, CAE (Ajinomoto, Teaneck, N.J.), cajeput oil, Cangzhu, capsicum frutescens extract, caraway oil, cascarilla bark, cedarleaf oil, chamomille, chaparral, chlorhexidine gluconate, chlorophenesin, chlorxylenol, cinnamon oil, citronella oil, clove oil, Crinipan AD (Climbazole), 2,3-dihydro-farnesol, dehydroacetic acid and its salts, dill seed oil, Dowicil 200 (Dow Chemical, Midland, Mich.), echinacea, elenolic acid, epimedium, ethyl paraben, Fo-Ti, galbanum, garden bumet, Germall 115 and Germall II (ISP-Sutton Labs, Wayne, N.J.), German chamomile oil, giant knotweed, Glydant (Lonza, Fairlawn, N.J.), Glydant Plus (Lonza), grapefruit seed oil, 1,6 hexanediol, hexamidine diisethionate, hinokitiol, honey, honeysuckle flower, hops, immortelle, iodopropynyl butyl carbamide (Lonza), isobutyl paraben, isopropyl paraben, JM Acticare (Microbial Systems International, Nottingham, NG), juniper berries, Kathon CG (Rohm and Haas, Philadelphia, Pa.), kojic acid, labdanum, lavender, lemon balm oil, lemon grass, methyl paraben, mint, mume, mustard, myrrh, neem seed oil, ortho phenyl phenol, olive leaf extract (Bio Botanica), parsley, patchouli oil, peony root, 1,2 pentandiol, Phenonip (Nipa Labs, Wilmington, Del.), phenoxyethanol, phytosphingosine, pine needle oil, Planservative (Campo Research), propyl paraben, purslane, quillaira, rhubarb, rose geranium oil, rosemary, sage, salicylic acid, sassafras, savory, sichuan lovage, sodium meta bisulfate, sodium sulfite, Sopholiance (Soliance, Compiegne, France), sorbic acid and its salts, sphingosine, stevia, storax, sucrose esters, tannic acid, tea, tea tree oil (cajeput oil), thyme, triclosan, triclocarban, tropolone, turpentine, umbelliferone (antifungal), yucca, and combinations thereof.

Suitable non-steroid cosmetic soothing agents include, for example, acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucolic acid, absinthium, acacia, aescin, alder buckthorn extract, allantoin, aloe, APT (Centerchem), arnica, astragalus, astragalus root extract, azulene, Baicalin SR 15 (Barnet Products Dist.), baikal skullcap, baizhu, balsam canada, bee pollen, Biophytex (Laboratories Serobiologiques), bisabolol, black cohosh, black cohosh extract blue cohosh, blue cohosh extract, boneset, borage, borage oil, bradykinin antagonists, bromelain, calendula, calendula extract, Canadian Willowbark Extract (Fytokem), candelilla wax, Cangzhu, canola phytosterols, capsicum, carboxypeptidase, celery seed, celery stem extract, Centaurium (Sederma), centaury extract, chamazulene, chamomile, chamomile extract, chaparral, chaste tree, chaste tree extract, chickweed, chicory root, chicory root extract, chirata, chishao, colloidal oatmeal, comfrey, comfrey extract, Cromoist CM Glucan (Croda), darutoside, dehurian angelica, devil's claw, divalent metals (such as, magnesium, strontium, and manganese), doggrass, dogwood, Eashave (Pentapharm), eleuthero, Elhibin (Pentapharm), Enteline 2 (Secma), ephedra, epimedium, esculoside; ethacrynic acid, evening primrose, eyebright, Extract LE-100 (Sino Lion), Fangfeng, feverfew, ficin, forsythia fruit, Fytosterol 85 (Fytokem), ganoderma, gaoben, Gatuline A (Gattefosse), gentian, germanium extract, gingko bilboa extract, ginkgo, ginseng extract, goldenseal, gorgonian extract, gotu kola, grape fruit extract, guaiac wood oil, guggal extract, helenalin esters, henna, honeysuckle flower, horehound extract, horsechestnut, horsetail, huzhang, hypericum, ichthyol, immortelle, ipecac, job's tears, jujube, kola extract, Lanachrys 28 (Lana Tech), lemon oil, lianqiao, licorice root, ligusticum, ligustrum, lovage root, luffa, mace, magnolia flower, manjistha extract, margaspidin, matricin, melatonin, Microat IRC (Nurture), mints, mistletoe, Modulene (Seporga), mono or diglucosides of glabridin, mono or diglucosides of gentian, MTA (5′-deoxy-5′-methythioadenosine), mung bean extract, musk, N-methyl arginine, oat beta glucan, oat extract, orange, panthenol, papain, phenoxyacetic acid, peony bark, peony root, Phytoplenolin (Bio Botanica), phytosphingosine, Preregen (Pentapharm), purslane, Quench T (Centerchem), quillaia, red sage, rehmannia, rhubarb, rosemary, rosmarinic acid, royal jelly, rue, rutin, sandalwood, sanqi, sarsaparilla, saw palmetto, Sensiline (Silab), Siegesbeckia (Sederma), stearyl glycyrrhetinate, Stimutex (Pentapharm), storax, strontium nitrate, sweet birch oil, sweet woodruff, tagetes, tea extract, thyme extract, tienchi ginseng, tocopherol, tocopheryl acetate, triclosan, turmeric, urimei, ursolic acid, white pine bark, witch hazel xinyi, yarrow, yeast extract, yucca, and combinations thereof.

Suitable steroids include, for example, fluocinolone acetonide, hydrocortisone butyrate, hydrocortisone propionate, hydrocortisone valerate, prednicarbate, flumethasone pivolate, clocortolone pivolate, triamcinolone acetonide, prednicarbate, fluticasone propionate, flurandrenolide, mometasone furoate, desoximetasone, betamethasone, betamethasone dipropionate, betamethasone valerate, betamethasone propionate, betamethasone benzoate, diflorasone diacetate, fluocinonide, halcinoruide, amcinonide, halobetasol propionate, clobetasol propionate, and combinations thereof.

Suitable skin conditioning agents include, for example, mineral oil, petrolatum, vegetable oils (such as soybean or maleated soybean oil), dimethicone, dimethicone copolyol, cationic monomers and polymers (such as guar hydroxypropyl trimonium chloride and distearyl dimethyl ammonium chloride), and combinations thereof. Illustrative moisturizers are polyols such as sorbitol, glycerin, propylene glycol, ethylene glycol, polyethylene glycol, polypropylene glycol, 1,3-butane diol, hexylene glycol, isoprene glycol, xylitol, fructose, and combinations thereof.

Suitable buffers, neutralizing'agents and agents to adjust pH include, for example, ammonium hydroxide, citric acid, diisopropanolamine, hydrochloric acid, lactic acid, monobasic sodium phosphate, sodium citrate, sodium hydroxide, sodium phosphate, triethanolamine, trolamine, and combinations thereof.

Suitable emollients include, for example, caprylic/capric triglycerides, castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, gyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, squalane, steareth-2 or -100, stearic acid, stearyl alcohol, urea, and combinations thereof.

Suitable emulsifying agents include, for example, aluminum starch octenylsuccinate, ammonium hydroxide, amphoteric-9, beeswax, synthetic beeswax, carbomer 934, carbomer 934P, carbomer 940, ceteareth-20, ceteareth-30, cetearyl alcohol, ceteth 20, cetyl alcohol, cholesterol, cyclomethicone, diglycerides, dimethicone (e.g., dimethicone 350), disodium monooleamidosulfosuccinate, NF emulsifying wax, fatty acid pentaerythritol ester, glycerides, glyceryl monooleate, glyceryl monostearate, lanolin, lanolin alcohol, hydrogenated lanolin, magnesium stearate, mineral oil, monoglycerides, polyethylene glycol, PEG 100 stearate, polyethylene glycol 6000 distearate, polyethylene glycol 1000 monocetyl ether, polyethylene glycol monostearate, polyethylene glycol 400 monostearate, polyoxyethylene glycol fatty alcohol ethers, polyoxyl 20 cetostearyl ether, polyoxyl 40 stearate, polysorbate 20, polysorbate 40, polysorbate 60, polysorbate 80, polysorbates, PPG-26 oleate, propylene glycol stearate, quaternium-15, simethicone, sodium laureth sulfate, sodium lauryl sulfate, sorbitan esters, sorbitan monolaurate, sorbitan monooleate, sorbitan monopalmitate, sorbitan monostearate, sorbitan palmitate, sorbitan sesquioleate, steareth-2, steareth-100, stearic acid, stearyl alcohol, triethanolamine and trolamine.

Suitable emulsion stabilizers and viscosity builders include, for example, carbomer 934, carbomer 934P, carbomer 940, cetearyl alcohol, cetostearyl alcohol, cetyl alcohol, cetyl stearyl alcohol, dextrin, diglycerides, disodium edetate, edetate disodium, glycerides, glyceryl monostearate, glyceryl stearate, hydroxypropyl cellulose, monoglycerides, plasticized hydrocarbon gel, polyethylene glycol 300, polyethylene glycol 400, polyethylene glycol 1450, polyethylene glycol 8000, polyethylene glycols, propylene glycol stearate and stearyl alcohol.

Suitable humectants include, for example, glycerine, propylene glycol, sorbitol, urea, and combinations thereof.

Suitable odorants include, for example, hypoallergenic perfume, menthol, and combinations thereof.

Suitable preservatives, antioxidants, and chemical stabilizers include, for example, alcohol, benzyl alcohol, butylated hydroxyanisole, butylated hydroxytoluene, butylparaben, calcium acetate, castor oil, chlorocresol, 4-chloro-m-cresol, citric acid, disodium edetate, Dowicil 200 (Dow), edetate disodium, ethoxylated alcohol, ethyl alcohol, glycerin, Glydant Plus (Lonza), 1,2,6-hexanetriol, Kathon CG (Rohm & Haas), Liquid Germall Plus (ISP Sutton Labs), Liquipar (ISP Sutton Labs), methylparaben, parabens, potassium sorbate, propyl gallate, propylene glycol, propylparaben, sodium bisulfite, sodium citrate, sodium metabisulfite, sorbic acid, tannic acid, triglycerides of saturated fatty acids, Ucarcide (Union Carbide), zinc stearate, and combinations thereof.

Suitable solvents include, for example, alcohol, castor oil, diisopropyl adipate, ethoxylated alcohol, ethyl alcohol, fatty alcohol citrate, glycerin, 1,2,6-hexanetriol, hexylene glycol, isopropyl alcohol, isopropyl myristate, isopropyl palm itate, mineral oil, phosphoric acid, polyethylene glycol 300, polyethylene glycol 400, polyethylene glycol 1450, polyethylene glycol 8000, polyethylene glycol 1000 monocetyl ether, polyethylene glycol monostearate, polyethylene glycol 400 monostearate, polyethylene glycols, polyoxyl 20 cetostearyl ether, polyoxypropylene 15-stearyl ether, polysorbate 20, polysorbate 40, polysorbate 60, polysorbate 80, polysorbates, propylene carbonate, propylene glycol, purified water, and SD alcohol 40, triglycerides of saturated fatty acids, and combinations thereof.

Suitable thickening, stiffening and suspending agents include, for example, aluminum stearate, beeswax, synthetic beeswax, carbomer 934, carbomer 934P, carbomer 940, cetostearyl alcohol, cetyl alcohol, cetyl esters wax, dextrin, glyceryl monostearate, hydroxypropyl cellulose, kaolin, paraffin, petrolatum, polyethylene, propylene glycol stearate, starch, stearyl alcohol, wax, white wax, xanthan gum, bentonite, and combinations thereof.

Other optional agents may be added to the composition including, for example, aloe, arachis oil, benzoic acid, cocoa butter, coenzyme Q10, Q10, dimethicone, eucalyptus oil, resorcinol, retinol, retinyl palmitate, retinyl acetate, fennel extract, whey protein, ceramide, silicone, alpha-hydroxy acids, beta-hydroxy acids, sorbitol, vitamin A, vitamin B, vitamin C, vitamin D, vitamin E, and vitamin K. Unless otherwise indicated, the composition will generally contain less than about 5% by weight and typically less than about 1% by weight of the above-ingredients.

### Treatment and Administration

The present invention provides compositions that can be used treating a wide variety of dermatitis, including, for example, poison ivy, poison oak, poison sumac, diaper rash, eczema, lichen simplex chronicus, rashes, dermatoses, seborrheic dermatitis, psoriasis, atopic dermatitis, or the combination thereof.

The compositions are particularly useful for treating poison ivy, poison oak, and diaper rash.

The composition of the invention is applied topically to the involved area until it has healed. For example, for contact dermatitis, a composition containing one or more anti-histamines or a pharmaceutically acceptable salt thereof; one or more polysaccharides; and one or more Group 1, 2, or 13 metal hydroxides, is preferably administered two to four times a day for from one day to a week or more until healing occurs.

Concentrations, amounts, etc., of various components are often presented in a range format throughout this disclosure. The description in range format is merely for convenience and brevity and should not be construed as an inflexible limitation on the scope of the claimed invention. Accordingly, the description of a range should be considered to have specifically disclosed all the possible sub ranges as well as individual numerical values within that range. For example, description of a range such as 1% to 8% should be considered to have specifically disclosed sub ranges such as 1% to 7%, 2% to 8%, 2% to 6%, 3% to 6%, 4% to 8%, 3% to 8% etc., as well as individual numbers within that range, such as, 2%, 5%, 7% etc. This construction applies regardless of the breadth of the range and in all contexts throughout this disclosure.

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B, C, and D, and lastly E” shall be construed to mean step A must be first, step E must be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance fulfills the more than one functional requirement as specified by claim language.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, the claims referred to above including but not limited to any original claims.

The invention should now be illustrated with the following non-limiting examples.

### EXAMPLES

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

### Example 1

### Preparation of Topical Gel Formulation Including Diphenhydramine Hydrochloride

To a small vial was added about 30 ml of Equate Children's Allergy Relief medicine (distributed by Wal-Mart Stores, Inc, Bentonville, Ark. 72716 and packaged by Perrigo, Allegan, Mich. 49010, which contained the active ingredient of about 12.5 mg diphenhydramine hydrochloride/5 ml with the following inactive ingredients: citric acid, FD&C Red No. 33, FD&C Red No.40, flavor, glycerin, high fructose corn syrup, poloxamer 407, purified water, sodium benzoate, sodium chloride, sodium citrate, and sorbitol), about 30 ml of Equate Max Strength Liquid Antacid (also distributed by Wal-Mart Stores, Inc, Bentonville, Ark. 72716 and packaged by Perrigo, Allegan, Mich. 49010, which contained the active ingredients/5 ml: about 400 mg aluminum hydroxide, about 400 mg magnesium hydroxide, and about 40 mg Simethicone with the following inactive ingredients: buytlparaben, carboxymethylcellulose sodium, flavor, hypromellose, microcrystalline cellulose, potassium citrate, propylparaben, purified water, Simethicone emulsion, and sorbitol), about ¼ teaspoon of Xanthan gum powder, and about 36 grams of corn starch. The formulation was mixed completely to form smooth gel-type dispersion.

### Example 2

### Preparation of Topical Gel Formulation Including Loratadine

To a small vial was added about 30 ml of Equate Children's Allergy Relief Loratadine Oral medicine (distributed by Wal-Mart Stores, Inc, Bentonville, Ark. 72716 and packaged by Perrigo, Allegan, Mich. 49010, which contained the active ingredient of about 6.0 mg loratadine/5 ml with the following inactive ingredients: citric acid, FD&C Red No. 33, FD&C Red No.40, flavor, glycerin, high fructose corn syrup, poloxamer 407, purified water, sodium benzoate, sodium chloride, sodium citrate, and sorbitol), about 30 ml of Equate Max Strength Liquid Antacid (also distributed by Wal-Mart Stores, Inc, Bentonville, Ark. 72716 and packaged by Perrigo, Allegan, Mich. 49010, which contained the active ingredients/5 ml: about 400 mg aluminum hydroxide, about 400 mg magnesium hydroxide, and about 40 mg Simethicone with the following inactive ingredients: buytlparaben, carboxymethylcellulose sodium, flavor, hypromellose, microcrystalline cellulose, potassium citrate, propylparaben, purified water, Simethicone emulsion, and sorbitol), about ¼ A teaspoon Xanthan gum powder, and about 36 grams of corn starch. The formulation was mixed completely to form smooth gel-type dispersion.

### Example 3

### Preparation of Topical Gel Formulation Including Cetirizine Hydrochloride

To a small vial was added about 30 ml of Equate Children's Allergy Relief Cetirizine Hydrochloride Oral medicine (distributed by Wal-Mart Stores, Inc, Bentonville, Ark. 72716 and packaged by Perrigo, Allegan, Mich. 49010, which contained the active ingredient of about 6.0 mg Cetirizine Hydrochloride/5 ml with the following inactive ingredients: citric acid, FD&C Red No. 33, FD&C Red No.40, flavor, glycerin, high fructose corn syrup, poloxamer 407, purified water, sodium benzoate, sodium chloride, sodium citrate, and sorbitol), about 30 ml of Equate Max Strength Liquid Antacid (also distributed by Wal-Mart Stores, Inc, Bentonville, Ark. 72716 and packaged by Perrigo, Allegan, Mich. 49010, which contained the active ingredients/5 ml: about 400 mg aluminum hydroxide, about 400 mg magnesium hydroxide, and about 40 mg Simethicone with the following inactive ingredients: buytlparaben, carboxymethylcellulose sodium, flavor, hypromellose, microcrystalline cellulose, potassium citrate, propylparaben, purified water, Simethicone emulsion, and sorbitol), about ¼ A teaspoon Xanthan gum powder, and about 36 grams of corn starch. The formulation was mixed completely to form smooth gel-type dispersion.

### Example 4

### Test Results for Treatment of Diaper Rash

To a skin affected with severe diaper rash was applied a composition containing the flowing ingredients. After 24 hours, the relative performance of the compositions was rated on a scale of 0 (no affect) to 10 (very good affect).

| Item | Formulation | Test Score |
|---|---|---|
| 1 | Corn Starch | 2 |
| 2 | Equate Children's Allergy Relief | 3 |
| | Medicine | |
| 3 | Equate Max Strength Liquid | 2 |
| | Antacid | |
| 4 | Xanthan Gum | 0 |
| 5 | Corn Starch + Equate Children's | 3 |
| | Allergy Relief Medicine | |
| 6 | Corn Starch + Equate Max | 2 |
| | Strength Liquid Antacid | |
| 7 | Corn Starch + Xanthan Gum | 2 |
| 8 | Equate Children's Allergy Relief | 3 |
| | Medicine + Xanthan Gum | |
| 9 | Equate Max Strength Liquid | 2 |
| | Antacid + Xanthan Gum | |
| 10 | Equate Children's Allergy Relief | 8 |
| | Medicine + Equate Max Strength | |
| | Liquid Antacid | |
| 11 | Equate Children's Allergy Relief | 9 |
| | Medicine + Equate Max Strength | |
| | Liquid Antacid + Corn Starch | |
| 12 | Equate Children's Allergy Relief | 9 |
| | Medicine + Equate Max Strength | |
| | Liquid Antacid + Xanthan Gum | |
| 13 | Equate Children's Allergy Relief | 10 |
| | Medicine + Equate Max Strength | |
| | Liquid Antacid + Xanthan Gum + | |
| | Corn Starch | |
| 14 | Control | 0 |

### Example 5

### Test Results for Treatment of Poison Ivy

To a skin affected with severe poison ivy was applied a composition containing the flowing ingredients. After 24 hours, the relative performance of the compositions was rated on a scale of 0 (no affect) to 10 (very good affect).

| Item | Formulation | Test Score |
|---|---|---|
| 1 | Corn Starch | 2 |
| 2 | Equate Children's Allergy Relief | 3 |
| | Medicine | |
| 3 | Equate Max Strength Liquid | 2 |
| | Antacid | |
| 4 | Xanthan Gum | 0 |
| 5 | Corn Starch + Equate Children's | 3 |
| | Allergy Relief Medicine | |
| 6 | Corn Starch + Equate Max | 2 |
| | Strength Liquid Antacid | |
| 7 | Corn Starch + Xanthan Gum | 2 |
| 8 | Equate Children's Allergy Relief | 3 |
| | Medicine + Xanthan Gum | |
| 9 | Equate Max Strength Liquid | 2 |
| | Antacid + Xanthan Gum | |
| 10 | Equate Children's Allergy Relief | 8 |
| | Medicine + Equate Max Strength | |
| | Liquid Antacid | |
| 11 | Equate Children's Allergy Relief | 9 |
| | Medicine + Equate Max Strength | |
| | Liquid Antacid + Corn Starch | |
| 12 | Equate Children's Allergy Relief | 10 |
| | Medicine + Equate Max Strength | |
| | Liquid Antacid + Xanthan Gum | |
| 13 | Equate Children's Allergy Relief | |
| | Medicine + Equate Max Strength | |
| | Liquid Antacid + Xanthan Gum + | |
| | Corn Starch | |
| 14 | Control | 0 |

### Example 6

### Test Results for Treatment of Poison Oak

To a skin affected with severe poison oak was applied a composition containing the flowing ingredients. After 24 hours, the relative performance of the compositions was rated on a scale of 0 (no affect) to 10 (very good affect).

| Item | Formulation | Test Score |
|---|---|---|
| 1 | Com Starch | 2 |
| 2 | Equate Children's Allergy Relief | 3 |
| | Medicine | |
| 3 | Equate Max Strength Liquid | 2 |
| | Antacid | |
| 4 | Xanthan Gum | 0 |
| 5 | Corn Starch + Equate Children's | 3 |
| | Allergy Relief Medicine | |
| 6 | Corn Starch + Equate Max | 2 |
| | Strength Liquid Antacid | |
| 7 | Corn Starch + Xanthan Gum | 2 |
| 8 | Equate Children's Allergy Relief | 3 |
| | Medicine + Xanthan Gum | |
| 9 | Equate Max Strength Liquid | 2 |
| | Antacid + Xanthan Gum | |
| 10 | Equate Children's Allergy Relief | 8 |
| | Medicine + Equate Max Strength | |
| | Liquid Antacid | |
| 11 | Equate Children's Allergy Relief | 9 |
| | Medicine + Equate Max Strength | |
| | Liquid Antacid + Corn Starch | |
| 12 | Equate Children's Allergy Relief | 9 |
| | Medicine + Equate Max Strength | |
| | Liquid Antacid + Xanthan Gum | |
| 13 | Equate Children's Allergy Relief | 10 |
| | Medicine + Equate Max Strength | |
| | Liquid Antacid + Xanthan Gum + | |
| | Corn Starch | |
| 14 | Control | 0 |

What is claimed is:

1. A composition for topical treatment of dermatitis comprising:
one or more anti-histamines or a pharmaceutically acceptable salt thereof;
one or more polysaccharides; and
one or more Group 1, 2, or 13 metal hydroxides,
wherein the dermatitis is poison ivy, poison oak, poison sumac, diaper rash, eczema, lichen simplex chronicus, rashes, dermatoses, seborrheic dermatitis, psoriasis, atopic dermatitis, or the combination thereof.

2. The composition of claim 1, wherein the composition comprises a solution, spray, lotion, gel, cream, or ointment.

3. The composition of claim 1, wherein the composition comprises a gel.

4. The composition of claim 1, wherein the one or more anti-histamines or a pharmaceutically acceptable salt thereof each comprise brompheniramine, chlorpheniramine, debrompheniramine, dexchlorpheniramine, carbinoxamine, clemastine, diphenhydramine, pyrilamine, tripelennamine, tripolidine, methdilazine, bromodiphenhydramine, promethazine, azatadine, cyproheptadine, diphenylpyraline, doxylamine, trimeprazine, phenindamine, ketotifen, hydroxyzine, tazifylline, temelastine, meclizine, acrivastine, setastine, oxatomide, mequitazine, levocabastine, lodoxamide, rocastine, phenindamine, azelastine, and ebastine, fexofenadine, loratadine, descarboethoxy loratadine, astemizole, norastemizole, desmethylastemizole, cetirizine, acrivastine, and temelastine, or a combination thereof.

5. The composition of claim 4, wherein the one or more anti-histamines or a pharmaceutically acceptable salt thereof each comprise diphenhydramine hydrochloride.

6. The composition of claim 1, wherein the one or more polysaccharides each comprise starch, a gum, or a combination thereof.

7. The composition of claim 6, wherein the starch comprises corn starch, potato starch, wheat starch, tapioca starch, cassava starch, arrowroot starch, arracacha starch, buckwheat starch, barley starch, oat starch, millet starch, rye starch, banana starch, breadfruit starch, canna starch, colacassia starch, katakuri starch, kudzu starch, malanga starch, oca starach, sago starch, sorghum starch, sweet potato starch, taro starch, water chestnut starch, yam starch, fava starch, lentils starch, mung bean starch, pea starch, or a combination thereof.

8. The composition of claim 7, wherein the starch is corn starch.

9. The composition of claim 6, wherein the gum comprises xanthan gum, gum Arabic, guar gum, red gum, gum acacia, sweet gum, black gum, kauri gum, or a combination thereof.

10. The composition of claim 9, wherein the gum is xanthan gum.

11. The composition of claim 6, wherein the one or more polysaccharides comprises the combination of corn starch and xanthan gum.

12. The composition of claim 1, wherein the one or more Group 1, 2, or 13 metal hydroxides each comprise lithium hydroxide, sodium hydroxide, potassium hydroxide, magnesium hydroxide, calcium hydroxide, boron hydroxide, aluminum hydroxide, or a combination thereof.

13. The composition of claim 12, wherein the one or more Group 1, 2, or 13 metal hydroxides comprise the combination of magnesium hydroxide and aluminum hydroxide.

14. A composition for topical treatment of dermatitis comprising:
diphenhydramine hydrochloride;
corn starch;
xanthan gum powder;
aluminum hydroxide; and
magnesium hydroxide;
wherein the dermatitis is poison ivy, poison oak, poison sumac, diaper rash, eczema, lichen simplex chronicus, rashes, dermatoses, seborrheic dermatitis, psoriasis, atopic dermatitis, or the combination thereof.

15. The composition of claim 14, wherein the composition comprises a solution, spray, lotion, gel, cream, or ointment.

16. The composition of claim 15, wherein the composition comprises a gel.

17. A method of treating dermatitis comprising
applying to a skin suffering from dermatitis a composition comprising
one or more anti-histamines or a pharmaceutically acceptable salt thereof;
one or more polysaccharides; and
one or more Group 1, 2, or 13 metal hydroxides,
wherein the dermatitis is poison ivy, poison oak, poison sumac, diaper rash, eczema, lichen simplex chronicus, rashes, dermatoses, seborrheic dermatitis, psoriasis, atopic dermatitis, or the combination thereof.

18. The method of claim 17, wherein the composition comprises a solution, spray, lotion, gel, cream, or ointment.

19. The method of claim 18, wherein the composition comprises a gel.

20. The method of claim 17, wherein the one or more anti-histamines or a pharmaceutically acceptable salt thereof each comprise diphenhydramine hydrochloride, wherein the one or more polysaccharides comprises the combination of corn starch and xanthan gum, and wherein the one or more Group 1, 2, or 13 metal hydroxides comprise the combination of magnesium hydroxide and aluminum hydroxide.
